(19) United States
(12) Patent Application Publication
(10) Pub. No.: US 2019/0329171 A1
(43) Pub. Date: Oct. 31, 2019

(54) FILTER MEDIUM, FILTER ELEMENT PROVIDED WITH SAME, AND METHOD FOR MANUFACTURING FILTER MEDIUM

(71) Applicant: FIL CORPORATION CO, LTD., Hadano-shi, Kanagawa (JP)

(72) Inventor: Tomoaki KANNO, Hadano-shi, Kanagawa (JP)

(73) Assignee: FIL CORPORATION CO, LTD., Hadano-shi, Kanagawa (JP)

(21) Appl. No.: 16/462,221

(22) PCT Filed: Dec. 7, 2017
(86) PCT No.: PCT/JP2017/043954

§ 371 Date: May 18, 2019

(30) Foreign Application Priority Data
Dec. 14, 2016 (JP) 2016-241867

(51) Int. Cl. B01D 46/52; B01D 46/00; B01D 53/04; B01D 39/16; C02F 1/28; C02F 1/00; B32B 3/28; B32B 5/02; B32B 5/26; B32B 7/14; B32B 37/06; B32B 37/10; B32B 37/12; B32B 37/18; B32B 38/00; A61L 9/16

(52) U.S. Cl. CPC B01D 46/522; A61L 2209/14; B01D 46/0001; B01D 53/04; B01D 39/163; C02F 1/283; C02F 1/001; B01D 46/0038; B32B 3/28; B32B 5/022; B32B 5/26; B32B 7/14; B32B 37/06; B32B 37/10; B32B 37/1207; B32B 37/182; B32B 38/0012; A61L 9/16; B01D 2253/102; B01D 2257/90; B32B 2255/02; B32B 2255/10; B32B 2255/26; B32B 2255/20; B32B 2264/108; B32B 2305/20; B32B 2305/72; B32B 2307/724; B32B 2307/726; B32B 2307/758; B32B 2313/04; B32B 2367/00; B32B 2250/02; B32B 2250/20; B32B 2250/244; B32B 2250/40; B32B 2262/0276; C02F 2303/02; B01D 46/0036

(57) ABSTRACT

To provide a filter medium that has small repulsion in pleating processing, has excellent air and water permeation resistance and filter performance, and has low pressure loss even when an adsorbent loading amount of the filter medium to be subjected to pleating processing is large, and to provide a filter element provided with the filter medium, and a method for manufacturing the filter medium. The filter medium includes an adsorptive layer interposed between air permeable substrates. At least one of the air permeable substrates has a folding streak on the inner face side thereof.

### TECHNICAL FIELD

The present invention relates to a filter medium having an adsorptive layer, a filter element provided with the same, and a method for manufacturing a filter medium.

### BACKGROUND ART

There are various types of pollutants in air, which are composed of polar gasses such as hydrogen sulfide, ammonia, aldehyde, and acetic acid, and low-polar gases such as benzene, toluene, and styrene.

Recently, in the field of filters for air controlling use, air conditioning use, automobile use, and semiconductor use, and so on, as a deodorant that removes these pollutants in air, a porous material of activated carbon, silica gel, or an ion exchange resin is often used. In rooms of houses, an air purifier that houses an air-conditioning filter is installed for the purpose of removing dust, toxic gases, etc., and to outside air intake ports of architectures, buildings, etc., an air-conditioning filter is attached, and clean air is taken in.

The air-conditioning filter to be used therefor is composed of a filter medium that is folded in a zigzag manner by subjecting a long non-woven fabric to folding processing (pleating processing) into a pleated shape, a space holding member (a corrugated aluminum material separator or a bead adhesive sandwiched between filter medium) sandwiched in a space between folds of the filter medium, and a filter frame (frame body) in which this filter medium is attached to an inner face resin with a sealing material of a synthetic resin (see, for example, PTL 1).

As a filter medium having adsorption performance to be used for such an air-conditioning filter, a method in which an activated carbon sheet is formed by sandwiching activated carbon between two substrate sheets (see, for example, PTLs 2 and 3), a material obtained by fixing and holding an adsorbent in a particulate form that adsorbs a material to be adsorbed to an intermediate substrate such as a net or a non-woven fabric, and superimposing non-woven fabrics for dust removal on both faces thereof and integrally joining these (see, for example, PTL 4), and a filter obtained by molding a deodorant and a dust removing material into a desired shape using a binder (see, for example, PTL 5) have been proposed. As a method for fixing an adsorbent, a method using an emulsion-based adhesive or using a hot-melt-type binder is known.

However, in a filter element such as a filter unit or a cartridge filter provided with a filter medium subjected to pleating processing, in order to enhance the deodorization performance, it is necessary to increase an adsorbent loading amount (the weight of an adsorbent per unit area). However, when the adsorbent loading amount is increased, the pressure loss is increased, and also the thickness of the filter medium is increased, and therefore, it becomes difficult to perform pleating processing of the filter medium. As a result, the repulsion of the folded filter medium is increased, and the number of folded pleats housed in the frame body is decreased, or the top of the folded pleat is rounded, and there is a problem that the structure pressure loss of the filter unit and the cartridge filter is increased.

A method for alternately forming streaks in pleating processing of a sheet-shaped filter medium (see, for example, PTL 6) has also been proposed. However, when an adsorbent loading amount is high, the rigidity of the filter medium is increased, and there is a problem that a crack occurs during pleating processing.

Further, a filter medium can also be applied to a cartridge filter for water treatment use. The cartridge filter is used in various fields such as water filtration for purified water to be used particularly in the field of pharmaceutical industry, the field of electronic industry, etc., filtration in a step of manufacturing an alcohol beverage in the field of food industry, and filtration for a painting agent in automobile industry.

However, it is often used separately for removal of ionic impurities dissolved in water such as removal of residual chlorine and removal of organic materials, and removal of particulate impurities such as removal of fine particles and removal bacteria.

A cartridge filter using an adsorbent is used in a water purifier using tap water as raw water or is used for the purpose of removal of residual chlorine and removal of organic materials from water in a step of manufacturing pure water or ultrapure water or the like in food industry, semiconductor industry, and so on, and a pleated filter body can increase the filtration area in the cartridge.

Also in the cartridge filter, in order to enhance the deodorization performance, or to also achieve removal of particulate impurities, it is necessary to increase the adsorbent loading amount. However, when the adsorbent loading amount is increased, due to an increase in the thickness of the filter medium, it becomes difficult to perform pleating processing of the filter medium, and the repulsion of the folded filter medium is increased. As a result, the number of folded pleats housed in a given frame is decreased, or the top of the folded pleat is rounded, and there is a problem that the structure pressure loss is increased.

### CITATION LIST

### Patent Literature

PTL 1:JP-A-2002-361016

PTL 2:JP-A-S61-119269

PTL 3:JP-A-2004-50151

PTL 4:JP-A-2002-17832

PTL 5:JP-A-H8-117524

PTL 6:JP-A-2003-265910

### SUMMARY OF INVENTION

### Technical Problem

The present invention has been made in view of such circumstances, and an object thereof is to provide a filter medium that has small repulsion in pleating processing, has excellent air and water permeation resistance and filter performance, and has low pressure loss even when an adsorbent loading amount of the filter medium to be subjected to pleating processing is large, a filter element provided with the same, and a method for manufacturing a filter medium.

### Solution to Problem

In order to achieve the above object, a filter medium of the present invention is a filter medium including an adsorptive layer interposed between air permeable substrates, wherein at least one of the air permeable substrates has folding a streak on an inner face side thereof as described in claim 1.

Further, a filter medium described in claim 2 is configured such that in the filter medium described in claim 1, the adsorptive layer is formed by adhering and solidifying an adsorbent with a thermal adhesive.

Further, a filter medium described in claim 3 is configured such that in the filter medium described in claim 1 or 2, both faces of the adsorptive layer are adhered to and integrated with the air permeable substrates.

Further, a filter medium described in claim 4 is configured such that in the filter medium described in any one of claims 1 to 3, a plurality of adsorptive layers are provided.

Further, a filter medium described in claim 5 is configured such that in the filter medium described in any one of claims 1 to 4, at least one of the air permeable substrates has a dust collecting function.

Further, a filter element of the present invention includes the filter medium described in any one of claims 1 to 5 as described in claim 6.

Further, a filter element described in claim 7 is configured such that in the filter element described in claim 6, a space holding member is sandwiched in a space between folds of the folded filter medium.

Further, a filter element described in claim 8 is configured such that in the filter element described in claim 6, the folded filter medium is subjected to corrugating processing.

A method for manufacturing a filter medium of the present invention includes a step of spraying and depositing an adsorbent and a thermal adhesive on a surface of a first air permeable substrate, thereby forming an adsorptive layer, a step of forming a streak along a width direction of the first air permeable substrate from an upper side of the adsorptive layer, a step of covering the first air permeable substrate with a second air permeable substrate so as to sandwich the adsorptive layer, and a step of heat-pressing the stacked first and second air permeable substrates, thereby curing the thermal adhesive as described in claim 9.

### Advantageous Effects of Invention

In the filter medium, the filter element, and the method for manufacturing a filter medium of the present invention, even if the adsorbent loading amount of the filter medium to be subjected to pleating processing is large, repulsion in pleating processing is small, the air and water permeation resistance and the filter performance are excellent, and low pressure loss can be realized.

### BRIEF DESCRIPTION OF DRAWINGS

FIG. 1 is an explanatory view showing a step of manufacturing a filter medium of this embodiment.

### DESCRIPTION OF EMBODIMENTS

Embodiments of a filter medium, a filter element provided with the same, and a method for manufacturing a filter medium according to the present invention will be described.

The filter medium is configured to interpose an adsorptive layer between air permeable substrates.

Further, the filter medium is provided with folding streaks on the inner face side of at least one of the air permeable substrates.

The adsorptive layer is a layer formed by adhering and solidifying an adsorbent with a thermal adhesive, and one face or both faces of the adsorptive layer is/are adhered to and integrated with the air permeable substrate. A plurality of adsorptive layers may be provided, and in such a case, the filter medium has a necessary number of air permeable substrates for sandwiching the adsorptive layers. For example, a plurality of adsorptive layers may be provided by stacking two sheets of the filter medium in which the adsorptive layer is interposed between a pair of air permeable substrates. Further, a plurality of adsorptive layers may be provided by alternately sandwiching the adsorptive layer and the air permeable substrate between a pair of air permeable substrates.

At least one of the air permeable substrates may have a dust collecting function.

The filter element is provided with the above-mentioned filter medium, and is, for example, a filter unit formed by housing the filter medium subjected to folding processing into a pleated shape (subjected to pleating processing) by being folded in a zigzag manner in a frame body, or a cartridge filter formed by circularly winding the filter medium around a core tube.

In the filter medium of the filter element, a space holding member may be sandwiched in a space between folds formed by folding. The space holding member is provided for ensuring an effective filtration area of the filter medium. The space holding member is, for example, a separator made of aluminum or a resin formed into a corrugated shape, a stabilizer in a comb-like shape, a resin bead adhered to the rear and front faces of the filter medium orthogonally to the pleat (a folding streak direction), or the like.

Alternatively, in the filter medium of the filter element, a filter medium provided with streaks can also be subjected to corrugating processing into a corrugated shape without using a space holding member. In such a case, since a space holding member is not used, the filter can be manufactured at low cost, and also the filter medium is processed into a corrugated shape, and therefore, the filter medium to be housed in the frame body is increased, and a filter having a long service life and low pressure loss can be formed. In the corrugating processing, generally, a filter medium is formed into a corrugated shape by being passed through a pair of gears, and the height of the corrugated shape can be controlled by the module of the gear to be used, and there are a spur gear, a helical gear, and the like.

As the air permeable substrate, a paper, a woven fabric, a non-woven fabric, and the like can be exemplified, and a non-woven fabric is preferred from the economical viewpoint.

As the non-woven fabric, a synthetic fiber such as a polyamide-based fiber, a polyester-based fiber, a polyurethane-based fiber, a polyvinyl alcohol-based fiber, a polyvinylidene chloride-based fiber, a polyvinyl chloride-based fiber, a polyacrylonitrile-based fiber, a polyolefin-based fiber, or a phenol-based fiber, an inorganic fiber such as a glass fiber, a metal fiber, an alumina fiber, or an activated carbon fiber, a natural fiber such as a wood pulp or a cotton linter pulp, a regenerated fiber, or the like is used.

A method for manufacturing the above-mentioned non-woven fabric is not particularly limited, and a dry method, a wet method, a melt blowing method, a spun bonding method, a needle punching method, a thermal bonding method, or the like can be used according to purpose or use.

As the adsorbent for forming the adsorptive layer, a powdery or particulate adsorbent can be used. As such a powdery or particulate adsorbent, an ion exchange resin, particulate activated carbon, silica gel, activated alumina, and catalyst particles are preferred. Examples of the catalyst particles that catalytically decompose an organic material or the like include metal simple substances such as iron, manganese, copper, aluminum, magnesium, zinc, nickel, cobalt, platinum, ruthenium, and rhodium, metal oxides or metal chlorides thereof, titanium oxide, and phthalocyanine. These powdery or particulate adsorbents may be used alone or two or more types thereof may be used in combination.

Further, the powdery or particulate adsorbent may be, for example, a material in which a chemical deodorant is adhered to the surface of the powdery or particulate adsorbent such as impregnated activated carbon. Examples of a chemical adsorbent for an aldehyde-based gas or an acidic gas such as NOx, SOx, or acetic acid to be adhered to impregnated activated carbon include alkali metal carbonates such as potassium carbonate, potassium hydrogen carbonate, sodium carbonate, and sodium hydrogen carbonate, alkali metal hydroxides such as sodium hydroxide and potassium hydroxide, amine compounds such as ethanolamine, hexamethylene diamine, methylamine, piperazine, aniline, p-anisidine, sulfanilic acid, and aminobenzoic acid and salts thereof, imines or imino compounds such as polyethyleneimine and iminodiethanol and salts thereof, guanidine-based compounds and salts thereof, L-arginine, methylamine hydrochlorides, semicarbazide hydrochlorides, hydrazine, hydroxylamine sulfate, and permanganate. Further, examples of a chemical adsorbent for an alkaline gas such as ammonia, an amine, or pyridine include organic acids and inorganic acids such as hydrochloric acid, sulfuric acid, nitric acid, malic acid, citric acid, and ascorbic acid.

Incidentally, these chemical adsorbents may be impregnated into the air permeable substrate or the air permeable substrate previously impregnated therewith may be used.

The average particle diameter of the powdery or particulate adsorbent is preferably within a range of about 4 to 200 mesh. This is because when the average particle diameter of the powdery or particulate adsorbent is large, the filter becomes thick and pleating processing becomes difficult, and when it is too small, the pressure loss is increased.

As the thermal adhesive, a hot-melt resin can be used. The using amount of the hot-melt resin is set to preferably 10 to 50%, more preferably 20 to 40% with respect to the weight of the powdery or particulate adsorbent to be used. When it is less than 10%, adhesion is poor, and the powdery or particulate adsorbent is not fixed to the air permeable substrate such as a non-woven fabric, and is likely to fall off from the air permeable substrate, and the pleated shape of the filter medium is lost. On the other hand, when it is more than 50%, most of the surface of the powdery or particulate adsorbent is covered with the hot-melt resin, and the deodorization performance or the like is deteriorated, and also the adsorptive layer is hardened more than necessary, and there is a fear that deterioration of processability during pleating processing is caused, or the hot-melt resin during melting is absorbed by the air permeable substrate and the air permeability of the air permeable substrate is deteriorated, or it exhibits stain-like appearance.

The average particle diameter of the hot-melt resin is preferably from about 10 to 400 mesh. When the average particle diameter of the hot-melt resin is larger than 10 mesh, a sufficient adhesive force is not obtained, and when it is smaller than 400 mesh, it causes an increase in air permeation resistance.

In order to impart antibacterial activity, antifungal activity, antiviral activity, flame retardancy, or another function to the air permeable substrate or the adsorbent, in the air permeable substrate itself or the hot-melt resin, an agent such as an antibacterial agent, an antiviral agent, a flame retardant, or a functional agent may be incorporated or mixed in advance. Further, such an agent may be impregnated into or fixed to the powdery or particulate adsorbent by a method and in an amount which do not deteriorate the original performance thereof, or may be adhered thereto by applying or the like the agent to the air permeable substrate.

To the air permeable substrate, a functionality other than the above-mentioned functionalities, for example, an electret property may be imparted. The electret property is used for collecting micron-sized dust such as bacteria or pollens by an air filter medium, and an electret melt-blown non-woven fabric is used. An electret processing method for imparting an electret property is not particularly limited, and for example, a known method such as a corona discharge method or a frictional charging method may be applied to a melt-blown non-woven fabric sheet. As the melt-blown non-woven fabric, a material having a high electrical resistivity such as polypropylene, polyethylene, polystyrene, polybutylene terephthalate, or polytetrafluoroethylene is preferably used for further obtaining collecting performance, and a composite product or a stacked product of each of the above-mentioned non-woven fabrics may be used.

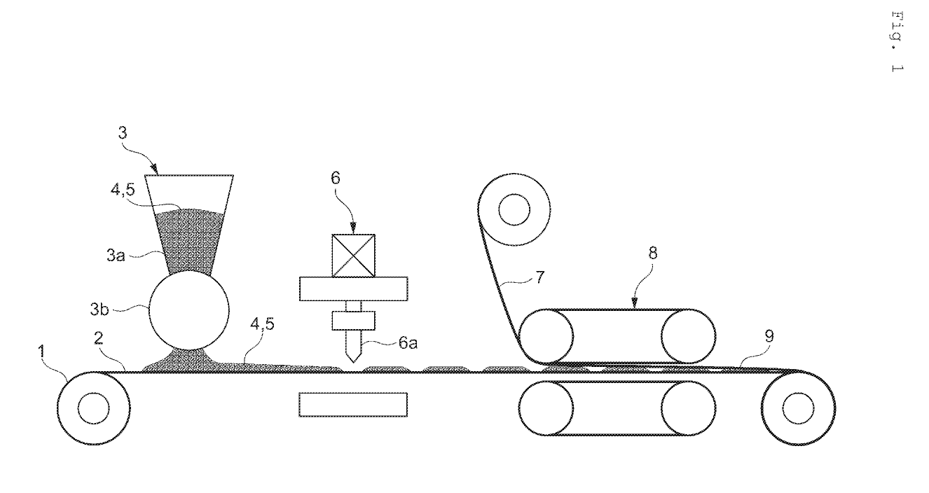

FIG. 1 is an explanatory view showing a step of manufacturing a filter medium of this embodiment.

An air permeable substrate 2 is wound into a roll shape and is sent in a flowing direction by a conveying device 1. On the surface of the air permeable substrate 2, an adsorbent 4 and a thermal adhesive 5 supplied from a hopper 3a of a roll-type spraying machine 3 are sprayed and deposited from a spraying roll 3b, whereby an adsorptive layer is formed. In the air permeable substrate 2, a streak is formed along the width direction with a blade 6a of a streak forming machine 6 from the upper side of the adsorptive layer. Thereafter, the air permeable substrate 2 is covered with another air permeable substrate 7 so as to sandwich the adsorptive layer. The stacked air permeable substrates 2 and 7 are conveyed to a heating pressing machine 8, and heat-pressed, whereby the thermal adhesive 5 is cured. Thereafter, the stacked air permeable substrates 2 and 7 are wound into a role shape as a filter medium 9.

The roll-type spraying machine 3 is composed of the hopper 3a storing a powder, and the spraying roll 3b supported rotatably by a lower portion of this hopper 3a. The method for spraying the powdery or particulate adsorbent onto the air permeable substrate 2 is not limited to the above-mentioned method and may be any as long as it is a conventionally known spraying method.

The streak forming machine 6 is composed of a driving mechanism such as a hydraulic cylinder, a vertical lift table, the blade 6a attached to the lift table, a blade receiving table having the same shape as the blade 6a or a blade receiving table having a flat table shape like a Thomson die that performs punching and cutting into a predetermined shape. The blade 6a vertically moves up and down from the upper side of the adsorptive layer with respect to a traveling sheet and forms a groove (streak) in the adsorptive layer sprayed and deposited before solidification and the air permeable substrate 2 with the edge of the blade 6a. The thus formed groove becomes a folding streak for pleating processing of the filter medium as solidifying the adsorptive layer. The interval of the grooves is arbitrarily adjusted by a folding width at which the filter medium is folded. The width and the depth of the groove can be adjusted according to the amount of the adsorbent 4 to be sprayed and deposited in consideration of the repulsion when folding or the structure pressure loss.

Further, as the shape of the blade 6a, not only a straight line shape, but also a zigzag shape, a corrugated shape, or the like may be adopted. When it is a zigzag shape, a corrugated shape, or the like, the area of the filter medium housed in the filter frame is increased, and also a space between the folded filter medium can be maintained even if a separator or a bead adhesive is not provided, and thus, it is economical.

Incidentally, in place of streak formation by a striping system performed using the streak forming machine 6, a rotary system in which a role with grooves at fixed intervals and a roll with projections at the same intervals rotate on the upper side and the lower side and form streaks while sandwiching the air permeable substrate 2 therebetween may be adopted.

The heating pressing machine 8 has a pair of flat heating belts and continuously performs heating for a given time at a given temperature and a given pressure.

According to this, the adsorbent 4 is fixed to the air permeable substrates 2 and 7 through the thermal adhesive 5. The heating pressing machine 8 may employ a known roll-to-roll pressing method in place of the above-mentioned heating pressing method.

As a method for performing pleating processing of the filter medium after the above-mentioned manufacturing step, for example, a method using a known pleating molding machine such as a reciprocating pleating molding machine that performs pleating processing of the filter medium by alternate movement of blades disposed one by one on the upper side and the lower side can be used.

The filter medium and the filter element provided with the same described above are manufactured by solidifying the adsorptive layer of the filter medium after forming grooves as streaks for performing pleating processing at a predetermined folding width. According to this, even if the adsorbent loading amount (the weight of the adsorbent per unit area) is increased for enhancing the performance of deodorization and removal of particulate impurities and the thickness of the filter medium is increased, repulsion when the filter medium is subjected to pleating processing is small, and pleating processing becomes easy.

Further, when the filter medium is used in a filter unit which is one example of the filter element, the number of folded pleats housed in the frame body can be increased. As a result, the adsorption amount of the filter unit is increased, and the filter performance such as deodorization can be obtained. Further, the tops of the folded pleats become sharp (acute angle), and the structure pressure loss of the filter unit can be reduced.

### EXAMPLES

Next, specific examples of the filter medium and the filter element of the present invention will be described.

In the following Examples, the physical properties such as weight per unit area, thickness, and air permeability of the filter medium were measured according to the method described in JIS L 1096 “General testing methods for woven fabrics”.

### Example 1

With respect to coconut shell activated carbon having an average particle diameter of 32 to 60 mesh (Coconut shell activated carbon 3260, manufactured by Kuraray Co., Ltd.) as the adsorbent, a thermoplastic powder resin of low-density polyethylene having an average particle diameter of 40 mesh and a melting point of 105° C. (PR1050M, manufactured by Tokyo Printing Ink Mfg.

Co., Ltd.) as the thermal adhesive at a weight ratio of 25% was mixed. This mixed particulate material was sprayed onto a polyester spun bond non-woven fabric having a weight per unit area of 30 g/m² as the air permeable substrate, and thereafter, streaks were formed at 92-mm intervals at right angles to the length direction of the sprayed and deposited mixed particulate material. Further, a polyester spun bond non-woven fabric having a weight per unit area of 30 g/m² was superimposed thereon from above so as to sandwich the mixed particulate material. A heating treatment was performed at 155° C. for 75 seconds so as to solidify the adsorptive layer provided with folding streaks and also to adhere the adsorptive layer to the two polyester spun bond non-woven fabrics, whereby a filter medium for deodorization was obtained.

The amount of the fixed activated carbon was 600 g/m², the air permeability of the filter medium was 50 cc/cm²/s, and the thickness thereof was 1.7 mm.

The obtained filter medium was subjected to pleating processing using a pleating molding machine, and a corrugated separator made of PET was interposed in a space between folds, and the resulting material was inserted into an aluminum frame. By doing this, a filter unit having a pleat height of 92 mm, including 65 pleats, and having a size of 610 mm (W)×610 mm (H)×100 mm (D) was obtained.

In the filter unit, repulsion in folding was small, the top of the pleat of the folded filter medium was sharp, and the area of the filter medium was 7.2 m². When the pressure loss of the filter unit was measured, it was 16.6 Pa at a face velocity of 0.5 m/s.

When a 10 cm×10 cm-square block was cut out from this filter unit and a removal service life was measured at a toluene concentration of 20 ppm and a face velocity of 0.5 m/s using a toluene detector tube (manufactured by Gastec Corporation), the service life at a breakthrough of 90% was 6.6 hours.

### Example 2

Two sheets of the filter medium manufactured in Example 1 were stacked, and a filter unit with a size of 610 mm (W)×610 mm (H)×100 mm (D) was obtained in the same manner as in Example 1. The pleat height was 92 mm, and the number of pleats was 47. In the filter unit, repulsion in folding was small, the top of the pleat of the folded filter medium was sharp, and the area of the filter medium was 10.5 m². When the pressure loss of the filter unit was measured, it was 45.7 Pa at a face velocity of 0.5 m/s.

When a removal service life was measured for this filter unit in the same manner as in Example 1, the service life at a breakthrough of 90% was 13.5 hours.

### Example 3

A filter medium for deodorization of an activated carbon sheet was obtained in the same manner as in Example 1 except that as a polyester spun bond non-woven fabric on the side superimposed on a polyester spun bond non-woven fabric provided with streaks, which is one of the air permeable substrates, a material obtained by bonding a melt-blown non-woven fabric (MPECO4S, manufactured by Mitsui Chemicals, Incorporated) made of polypropylene and having a dust collecting function and a polyester spun bond non-woven fabric having a weight per unit area of 30 g/m² used in Example 1 was used, and the streak forming interval was set to 285 mm.

The amount of the fixed activated carbon was 300 g/m², the air permeability of the filter medium was 45 cc/cm²/s, and the thickness thereof was 1.3 mm.

The obtained filter medium was subjected to pleating processing using a pleating molding machine, and a corrugated separator made of PET was interposed in a space between folds, and the resulting material was inserted into an aluminum frame. By doing this, a filter unit having a pleat height of 285 mm, including 41 pleats, and having a size of 610 mm (W)×610 mm (H)×290 mm (D) was obtained.

In the filter unit, repulsion in folding was small, the top of the pleat of the folded filter medium was sharp, and the area of the filter medium was 14.1 m².

When the pressure loss of the filter unit was measured, it was 93.1 Pa at a face velocity of 2.5 m/s.

### Example 4

A filter medium for deodorization of a cation sheet was obtained in the same manner as in Example 1 except that an ion exchange resin (SK1BH, manufactured by Mitsubishi Chemical Corporation) as the adsorbent, and a polyester hot melt having a melting point of 125° C. (G120, manufactured by Tokyo Printing Ink Mfg. Co., Ltd.) as the thermal adhesive were used, and the streak forming interval was set to 45 mm.

The amount of the fixed cation exchange resin was 500 g/m², the air permeability of the filter medium was 80 cc/cm²/s, and the thickness thereof was 1.5 mm.

The obtained filter medium was subjected to pleating processing using a pleating molding machine, and a corrugated separator made of PET was interposed in a space between folds, and the resulting material was inserted into an aluminum frame. By doing this, a filter unit having a pleat height of 45 mm, including 75 pleats, and having a size of 610 mm (W)×610 mm (H)×50 mm (D) was obtained.

In the filter unit, repulsion in folding was small, the top of the pleat of the folded filter medium was sharp, and the area of the filter medium was 4.1 m². When the pressure loss of the filter unit was measured, it was 18.1 Pa at a face velocity of 0.5 m/s.

When a removal service life was measured for this filter unit in the same manner as in Example 1, the service life at a breakthrough of 90% was 5.4 hours.

### Example 5

A filter medium for deodorization was obtained in the same manner as in Example 1 except that the streak forming interval was set to 33 mm. The obtained filter medium was subjected to pleating processing using a pleating molding machine, whereby a pleated filter medium having a pleat height of 33 mm and including 29 pleats was obtained. This pleated filter medium was circularly wound around a porous core tube made of polypropylene having an outer diameter of 32 mm, whereby a cartridge filter having an outer diameter of 65 mm and a length of 250 mm was obtained.

In the cartridge filter, repulsion in folding was small, the top of the folded filter medium was sharp, and the area of the filter medium was 0.48 m² Comparative Example 1

A filter medium for deodorization was obtained in the same manner as in Example 1 except that streaks were not formed in the sprayed and deposited mixed particulate material.

The amount of the fixed activated carbon was 600 g/m² in the same manner as in Example 1, the air permeability of the filter medium was 50 cc/cm²/s, and the thickness thereof was 1.7 mm.

The obtained filter medium was subjected to pleating processing in the same manner as in Example 1, whereby a filter unit having a size of 610 mm (W)×610 mm (H)×10 mm (D) was obtained.

In this filter unit, the pleat height was 92 mm, however, the top of the pleat of the folded filter medium had a round shape, the heights of the pleats were not uniform, repulsion in folding of the filter medium was large, and the number of pleats which could be inserted into the aluminum frame was 60. The area of the filter medium was 6.6 m². When the pressure loss of the filter unit was measured, it was 24.8 Pa at a face velocity of 0.5 m/s.

When a removal service life was measured for this filter unit in the same manner as in Example 1, the service life at a breakthrough of 90% was 5.1 hours.

### Comparative Example 2

Two sheets of the filter medium manufactured in Comparative Example 1 were stacked, and a filter unit with a size of 610 mm (W)×610 mm (H)×100 mm (D) was obtained in the same manner as in Comparative Example 1.

In this filter unit, the pleat height was 92 mm, however, in the same manner as in Comparative Example 1, the top of the pleat of the folded filter medium had a round shape, the heights of the pleats were not uniform, repulsion in folding of the filter medium was large, and the number of pleats which could be inserted into the aluminum frame was 43. The area of the filter medium was 9.5 m². When the pressure loss of the filter unit was measured, it was 69.3 Pa at a face velocity of 0.5 m/s.

When a removal service life was measured for this filter unit in the same manner as in Example 1, the service life at a breakthrough of 90% was 11.2 hours.

### Comparative Example 3

A filter medium for deodorization was obtained in the same manner as in Example 3 except that streaks were not formed in the sprayed and deposited mixed particulate material.

The amount of the fixed activated carbon was 300 g/m², the air permeability of the filter medium was 45 cc/cm²/s, and the thickness thereof was 1.3 mm.

The obtained filter medium was subjected to pleating processing using a pleating molding machine, and a corrugated separator made of PET was interposed in a space between folds, and the resulting material was inserted into an aluminum frame. By doing this, a filter unit having a pleat height of 285 mm, including 35 pleats, and having a size of 610 mm (W)×610 mm (H)×290 mm (D) was obtained.

In this filter unit, the pleat height was 285 mm, however, in the same manner as in Comparative Example 1, the top of the pleat of the folded filter medium had a round shape, the heights of the pleats were not uniform, repulsion in folding of the filter medium was large, and the number of pleats which could be inserted into the aluminum frame was 35. The area of the filter medium was 12.1 m². When the pressure loss of the filter unit was measured, it was 130 Pa at a face velocity of 2.5 m/s.

### Comparative Example 4

A filter medium for deodorization of a cation sheet was obtained in the same manner as in Example 4 except that streaks were not formed in the sprayed and deposited mixed particulate material.

The amount of the fixed cation exchange resin was 500 g/m², the air permeability of the filter medium was 80 cc/cm²/s, and the thickness thereof was 1.5 mm.

The obtained filter medium was subjected to pleating processing in the same manner as in Example 4, whereby a filter unit having a size of 610 mm (W)×610 mm (H)×50 mm (D) was obtained.

In this filter unit, the pleat height was 45 mm, however, in the same manner as in Comparative Example 1, the top of the pleat of the folded filter medium had a round shape, the heights of the pleats were not uniform, repulsion in folding of the filter medium was large, and the number of pleats which could be inserted into the aluminum frame was 70. The area of the filter medium was 3.8 m². When the pressure loss of the filter unit was measured, it was 27.2 Pa at a face velocity of 0.5 m/s.

When a removal service life was measured for this filter unit in the same manner as in Example 1, the service life at a breakthrough of 90% was 3.8 hours.

### Comparative Example 5

A filter medium for deodorization was obtained in the same manner as in Example 5 except that streaks were not formed in the sprayed and deposited mixed particulate material. The obtained filter medium was subjected to pleating processing using a pleating molding machine, whereby a pleated filter medium having a pleat height of 33 mm and including 29 pleats was obtained. This filter medium was circularly wound around a porous core tube made of polypropylene having an outer diameter of 32 mm to try to obtain a cartridge filter having an outer diameter of 65 mm and a length of 250 mm. However, the heights of the pleats were not uniform, and repulsion in folding of the filter medium was large, and the filter medium could not be formed into a cartridge.

Various evaluation results for the filter units and the cartridge filters of Examples 1 to 5 and Comparative Examples 1 to 5 are shown in Table 1.

TABLE 1

| | | Air | | | | |
|---|---|---|---|---|---|---|
| | Amount of | permeability | Thickness | | | |
| | adsorbent of | of | of | Number of | Folding | Number |
| | filter medium | filter medium | filter medium | filter medium | width | of pleat |
| | Unit | | | | | |
| Items | g/m² | cc/cm²/s | mm | sheet | mm | pleat |
| Ex. 1 | 600 | 50 | 1.7 | 1 | 92 | 65 |
| Ex. 2 | 600 | 50 | 1.7 | 2 | 92 | 47 |
| Ex. 3 | 300 | 45 | 1.3 | 1 | 285 | 41 |
| Ex. 4 | 500 | 80 | 1.5 | 1 | 45 | 75 |
| Ex. 5 | 600 | 50 | 1.7 | 1 | 33 | 29 |
| Com. Ex. 1 | 600 | 50 | 1.7 | 1 | 92 | 60 |
| Com. Ex. 2 | 600 | 50 | 1.7 | 2 | 92 | 43 |
| Com. Ex. 3 | 300 | 45 | 1.3 | 1 | 285 | 35 |
| Com. Ex. 4 | 500 | 80 | 1.5 | 1 | 45 | 70 |
| Com. Ex. 5 | 600 | 50 | 1.7 | 1 | 33 | 29 |

| | | | | Pressure loss | |
|---|---|---|---|---|---|
| | | Uniform of | | (face velocity | Service life |
| | Area of | heights of | Shape of | 0.5 m/s, | at |
| | filter material | pleats | pleats top | 2.5 m/s in Ex. 3) | breakthrough |
| | Unit | | | | |
| Items | m² | — | — | Pa | hr |
| Ex. 1 | 7.2 | ◯ | sharp | 16.6 | 6.6 |
| Ex. 2 | 10.5 | ◯ | sharp | 45.7 | 13.5 |
| Ex. 3 | 14.1 | ◯ | sharp | 93.1 | — |
| Ex. 4 | 4.1 | ◯ | sharp | 18.1 | 5.4 |
| Ex. 5 | 0.48 | ◯ | sharp | — | — |
| Com. Ex. 1 | 6.6 | X | Round | 24.8 | 5.1 |
| Com. Ex. 2 | 9.5 | X | Round | 69.3 | 11.2 |
| Com. Ex. 3 | 12.1 | X | Round | 130 | — |
| Com. Ex. 4 | 3.8 | X | Round | 27.2 | 3.8 |
| Com. Ex. 5 | — | X | Round | — | — |

From Table 1, it is apparent that in the filter medium and the filter element of the present invention, even when the adsorbent loading amount of the filter medium to be subjected to pleating processing is large, repulsion in pleating processing is small, the air and water permeation resistance and the filter performance are excellent, and low pressure loss can be realized.

### Example 6

The filter medium obtained in Example 4 was subjected to corrugating processing with a thickness of 3 mm by being passed through a pair of spur gears, and the obtained filter medium was subjected to pleating processing using a pleating molding machine. In the pleating processing, pleating was performed for each streak, and the top of the folded pleat was sharp.

### Comparative Example 6

On the other hand, the filter medium obtained in Comparative Example 4 was subjected to pleating processing after corrugating processing in the same manner, however, the corrugated portion was crushed, the top of the pleat became flat, and pleating processing could not be performed.

In this manner, in the filter element of this Example, it could be confirmed that a filter element can be formed by subjecting a filter medium provided with streaks to corrugating processing into a corrugated shape without using a space holding member.

### REFERENCE SIGNS LIST

1 Conveying device
2, 7 Air permeable substrate
3 Roll-type spraying machine
3a Hopper
3b Spraying roll
4 Adsorbent
5 Thermal adhesive
6 Streak forming machine
6a Blade
8 Heating pressing machine
9 Filter medium

1: A filter medium comprising an adsorptive layer interposed between air permeable substrates, wherein at least one of the air permeable substrates has a folding streak on an inner face side thereof.

2: The filter medium according to claim 1 wherein the adsorptive layer is formed by adhering and solidifying an adsorbent with a thermal adhesive.

3: The filter medium according to claim 1 wherein both faces of the adsorptive layer are adhered to and integrated with the air permeable substrates.

4: The filter medium according to claim 1, wherein a plurality of adsorptive layers are provided.

5: The filter medium according to claim 1, wherein at least one of the air permeable substrates has a dust collecting function.

6: A filter element comprising the filter medium according to claim 1.

7: The filter element according to claim 6 further comprising a space holding member sandwiched in a space between folds of the folded filter medium.

8: The filter element according to claim 6 wherein the folded filter medium is subjected to corrugating processing.

9: A method for manufacturing a filter medium comprising:
a step of spraying and depositing an adsorbent and a thermal adhesive on a surface of a first air permeable substrate, thereby forming an adsorptive layer;
a step of forming a streak along a width direction of the first air permeable substrate from an upper side of the adsorptive layer;
a step of covering the first air permeable substrate with a second air permeable substrate so as to sandwich the adsorptive layer; and
a step of heat-pressing the stacked first and second air permeable substrates, thereby curing the thermal adhesive.
